(19) United States
(12) Patent Application Publication
(10) Pub. No.: US 2025/0027968 A1
(43) Pub. Date: Jan. 23, 2025

(54) DEVICE AND AUTOMATED LABORATORY MACHINE FOR INCUBATING MULTIPLE PATIENT SAMPLES USING MULTIPLE INCUBATION VESSELS

(71) Applicant: Euroimmun Medizinische Labordiagnostika AG, Luebeck (DE)

(72) Inventors: Kolja Petter, Luebeck (DE); Alexander Malzkuhn, Luebeck (DE); Björn Rehm, Luebeck (DE)

(73) Assignee: Euroimmun Medizinische Labordiagnostika AG, Luebeck (DE)

(21) Appl. No.: 18/777,967

(22) Filed: Jul. 19, 2024

(30) Foreign Application Priority Data
Jul. 19, 2023 (EP) 23186403.4

(51) Int. Cl. G01N 35/10; G01N 35/00

(52) U.S. Cl. CPC G01N 35/1065; G01N 35/00584; G01N 2035/00356

(57) ABSTRACT

A device incubates multiple patient samples using multiple incubation vessels, including a first unit having respective receptacles for respective incubation vessels, and a second unit having respective receptacles for respective incubation vessels. Both of the respective units are each movably mounted in their respective position along a common linear guide axis, and further include respective sensors for providing respective sensor signals, each of which indicates one of the respective positions, furthermore respective magnetic drive units for a respective change of a respective position, and furthermore a control unit designed to control the respective magnetic drive units on the basis of the respective sensor signals to cause the respective changes of the respective positions.

### CROSS-REFERENCE TO RELATED APPLICATIONS

This application claims the benefit of priority to European Patent Application No. 23186403.4, filed on Jul. 19, 2023, the entire content of which is hereby incorporated by reference herein.

### BACKGROUND

### Field of the Invention

The invention relates to a device and an automated laboratory machine for incubating multiple patient samples using multiple incubation vessels.

### Description of Related Art

For a multitude of technical methods in the fields of biotechnology, pharmacy, diagnostics and medicine, the aim is to incubate multiple or respective patient samples in respective incubation vessels. Incubation is understood here to mean contacting a patient sample with one or more liquids of a so-called assay or diagnostic assay. This requires not only contacting of the patient sample with one or more of such liquids of an assay for a short time, but also accommodation of the patient sample and further liquids by the corresponding incubation vessel for a certain period of time. In this connection, the incubation vessel is usually moved or shaken rhythmically to optimize mixing of the patient samples and such liquids. Furthermore, the incubation vessel may also preferably be exposed in this connection to a predetermined temperature, which then prevails within the incubation vessel.

For such purposes, it is desirable for logistical and economic reasons to increase the degree of automation of such incubation processes.

Known are devices in which a single incubation vessel is introduced into a holder and in which automated movement of the holder then simply also causes rhythmic movement or shaking of the incubation vessel.

### SUMMARY OF THE INVENTION

It is an object of the present invention to further increase the degree of automation, such that incubation of multiple patient samples in corresponding or respective incubation vessels can take place simultaneously at least to some extent.

The object of the invention is achieved by a device and by an automated laboratory machine described herein.

The device according to the invention for incubating multiple patient samples comprises a first unit having respective, i.e., multiple, receptacles for respective incubation vessels and a second unit having respective, i.e., multiple, receptacles for respective incubation vessels.

Both units are each movably mounted in their respective position along a common linear guide axis.

The device further comprises respective sensors for providing respective sensor signals, the respective sensor signals each indicating one of the respective positions of a respective unit.

Furthermore, the device comprises respective magnetic drives or magnetic drive units, each of which is designed to bring about or cause a respective change of a respective position of a respective unit.

Lastly, the device further comprises a control unit designed to control the respective magnetic drive units on the basis of the respective sensor signals to cause or bring about the respective changes of the respective positions.

In the device according to the invention, two units each have multiple incubation vessel receptacles, such that a plurality of incubation vessels can be exposed simultaneously to a rhythmic movement or a shaking process. Because both units are movably mounted along the common linear guide axis, said both units can move translationally only along said axis and, in particular, be made to oscillate only along said axis. In particular, the two units are thus mounted and guided in such a way that they have only one common translational degree of freedom along the common linear guide axis. This makes it possible in principle for the mass movements of the two units with the incubation vessels present therein to take place not in a disordered or chaotic manner, but simply just along the common linear guide axis, and so in principle these two mass movements can compensate each other, especially when the device is installed in an overall automated laboratory machine.

Because the control unit is aware of the respective positions of the respective units on the basis of the respective sensor signals of the respective sensors, the control unit can thus advantageously bring about a respective change of a respective position on the respective unit via the respective magnetic drive units. If only a single unit were used for incubation, a rhythmic movement of said single unit would have an effect on the overall automated laboratory machine, thus also potentially causing oscillation of other units in the automated laboratory machine. By providing two separate units which are movably mounted in the direction of the common linear guide axis, such a mass movement of a single unit can be compensated by a mass movement of the second unit. In particular, the control unit thus controls the magnetic drive units in such a way that a mass movement of the first unit is compensated by a mass movement of the second unit. This minimizes the overall mass movement of the overall mass of units and thus also minimizes a mechanical effect on the overall automated laboratory machine.

### DETAILED DESCRIPTION OF THE INVENTION

Advantageous embodiments of the invention are subject matter of the dependent embodiments and are more particularly elucidated in the description with reference in some cases to the figures.

Preferably, the two units are each mounted and guided along the common linear guide axis in such a way that they have only one common translational degree of freedom. This ensures that the mass movements of the units are effected only along the one linear guide axis or the one translational degree of freedom and that the control unit thus only has to be designed to control the mass movements of the respective units only along said translational degree of freedom or along said common linear guide axis such that the mass movements compensate each other to at least a certain degree.

Preferably, a respective unit is mechanically coupled to a common base plate via a respective spring unit. Here, a respective unit together with a respective spring unit preferably forms a respective spring-mass oscillation system. This configuration is advantageous because the control unit can thus cause each of the respective units to oscillate at a certain frequency via the respective magnetic drive units. Here, the control unit can make use of the frequency response of a respective spring-mass oscillation system and must preferably only generate drive energy via the magnetic drive units in such a way to compensate possible energy losses due to mechanical friction and/or deviations of the oscillation frequencies of the spring-mass oscillation systems from a target frequency. The control unit is thus preferably designed to cause especially the respective units to respectively oscillate at identical frequency via the respective magnetic drive units.

The control unit is further preferably designed to control the magnetic drive units such that the units are each maintained in oscillation with a respectively substantially identical oscillation amplitude in the direction of the linear guide axis or the translational degree of freedom. The oscillation amplitudes can also be referred to as maximum oscillation amplitudes.

In particular, the control unit is preferably designed to control the magnetic drive units by means of a feedback control principle on the basis of the sensor signals such that the resultant respective oscillation frequencies of the respective units are substantially identical and that the respective oscillation frequencies have a phase shift of substantially 180 degrees to each other. Particularly preferably, the resultant respective oscillation frequencies of the respective units are identical and have a phase shift of 180 degrees to each other. This configuration is especially advantageous because compensation of a mass movement of the first unit with respect to a mass movement of the second unit is then particularly optimized or made possible, thus particularly minimizing in particular an overall effect of vibrational forces of the two common units on an automated laboratory machine.

In particular, the control unit is designed to determine a respective current amplitude or oscillation amplitude and a respective oscillation frequency for a respective unit on the basis of a respective sensor signal and further in particular to adjust both units to a common oscillation amplitude and a common, identical target oscillation frequency. This achieves optimization of vibration compensation of the two units particularly well. The common oscillation amplitude can also be referred to as common maximum oscillation amplitude or as maximum target oscillation amplitude.

In particular, both units preferably have an identical mass in the unloaded state. In particular, the two spring units are designed so as to have an identical, common spring constant.

Preferably, both units with the respectively associated spring units comprise a respective spring-mass oscillation system with a respectively identical resonance frequency in the unloaded state, wherein a target oscillation frequency for feedback control by the control unit is below said resonance frequency. This configuration is advantageous because loading of one or both units with incubation vessels, in particular with liquids present in said vessels, increases a respective total mass of a respective unit, thus reducing a resonance frequency from the unloaded state of the system toward a somewhat lower resonance frequency. Because the target oscillation frequency for feedback control by the control unit is below the resonance frequency of one of the spring-mass oscillation systems in the unloaded state, this thus means that, in the loaded state of the units, the target oscillation frequency is close to a resonance frequency of the spring-mass oscillation systems in the loaded state of the units. In particular, the respective units with their respective spring units each form identical spring-mass oscillation systems.

Preferably, the control unit is further designed to perform a PI control based on a common, identical target oscillation frequency. Furthermore, the control unit is preferably designed to perform a PI control based on a common, identical target oscillation frequency and a common, identical maximum oscillation amplitude for both units, and in particular the respective oscillation frequencies of the respective units have a phase shift of 180 degrees to each other. This configuration is advantageous because PI controls are particularly stable.

Preferably, a respective unit has a respective heating unit. This configuration is advantageous because desired temperatures within the incubation vessels can thus be reached.

Further proposed is an automated laboratory machine comprising a device for incubation according to the invention. The automated laboratory machine further comprises a magazine containing a plurality of incubation vessels. Furthermore, the automated laboratory machine comprises a container containing patient sample liquid, a container containing buffer liquid, a container containing bead liquid, which in turn comprises beads coated with antigens or antibodies, and a container containing a label liquid comprising antibodies or antigens labeled with a chemiluminescence label. The automated laboratory machine is thus in particular an automated laboratory machine for processing a patient sample using a chemiluminescence assay for detection of a target antigen or a target antibody in the patient sample.

Furthermore, the automated laboratory machine comprises a pipetting unit designed to dispense at least a portion of the patient sample liquid, at least a portion of the buffer liquid, at least a portion of the bead liquid and at least a portion of the label liquid comprising the antibodies or antigens labeled with a chemiluminescence label into the incubation vessels and to aspirate them from the incubation vessels.

The automated laboratory machine further comprises a gripping unit designed to transfer incubation vessels from the magazine to the pipetting unit and to transfer them from the pipetting unit to respective receptacles, to remove them from the respective receptacles and to transfer them back to the pipetting unit. The automated laboratory machine according to the invention is advantageous because, for the purpose of processing a chemiluminescence assay using antibodies or antigens labeled with a chemiluminescence label as part of the assay, patient samples can be incubated or processed in respective incubation vessels in the two units in accordance with the chemiluminescence assay.

Preferably, further proposed is an automated laboratory machine having one of the embodiments of the device for incubation as disclosed herein.

The automated laboratory machine preferably further comprises a container containing an enzyme for implementation of a chemiluminescence reaction and further comprises a reading unit for detecting an optical signal of a chemiluminescence reaction. This configuration is advantageous because an optical signal of a chemiluminescence reaction can then be brought about and detected in an automated manner.

Preferably, the magnetic drives of the device are arranged such that beads present in the bead liquid are not substantially influenced magnetically by a magnetic field of the magnetic drives. This is advantageous because beads are usually magnetizable particles in the form of metallic beads that are not to be moved in a preferred direction or to a preferred location by the magnetic drives while mixing is being carried out in the course of incubation.

In the following, the invention will be more particularly elucidated on the basis of specific embodiments, without limitation of the general inventive concept, with reference to the figures. In the figures:

### BRIEF DESCRIPTION OF THE DRAWINGS

FIG. 1A shows a top view of an exemplary device according to the invention

FIG. 1B shows an exemplary detailed view of one unit of the device according to a preferred embodiment

FIG. 2 shows a schematic representation of the device according to a preferred embodiment

FIG. 3 shows further details of a preferred embodiment of the device

FIG. 4 shows a schematic diagram of a preferred embodiment of an automated laboratory machine according to the invention

FIG. 5A shows an interaction of a sensor with a magnet

FIG. 5B shows a family of characteristics with signals

FIGS. 6A and 6B show details of a gripping unit interacting with an incubation vessel according to a preferred embodiment

FIGS. 7A and 7B show details of receptacles for incubation vessels and an incubation vessel according to a preferred embodiment

FIGS. 8A to 8D show signals and frequency plots

FIG. 9A shows details of a preferred embodiment of a sub-control unit

FIG. 9B shows further details of a preferred embodiment of a control unit

FIG. 10 shows details of an exemplary feedback control by a further sub-control unit

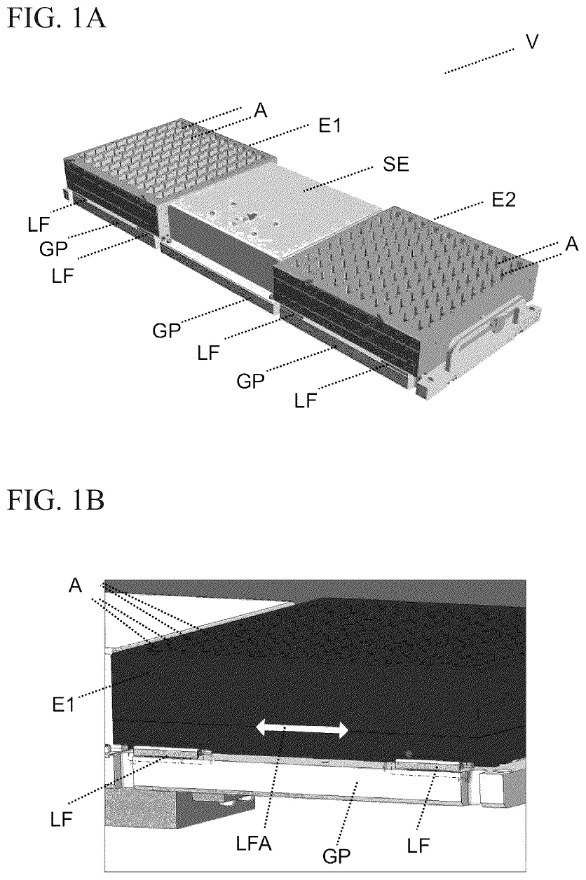

FIG. 1A shows a device V comprising two units E1, E2 having respective receptacles A for respective incubation vessels. The units E1, E2 are movably mounted along a common linear guide axis LFA —see FIG. 1B—by means of linear guides LF. Further shown is a control unit SE. The units E1, E2 are mounted on a common base plate GP by means of the linear guide elements LF.

In relation to this, FIG. 1B shows a detailed view of the unit E1 and of linear guide elements LF, surrounded by a dash-dotted frame, and the base plate GP.

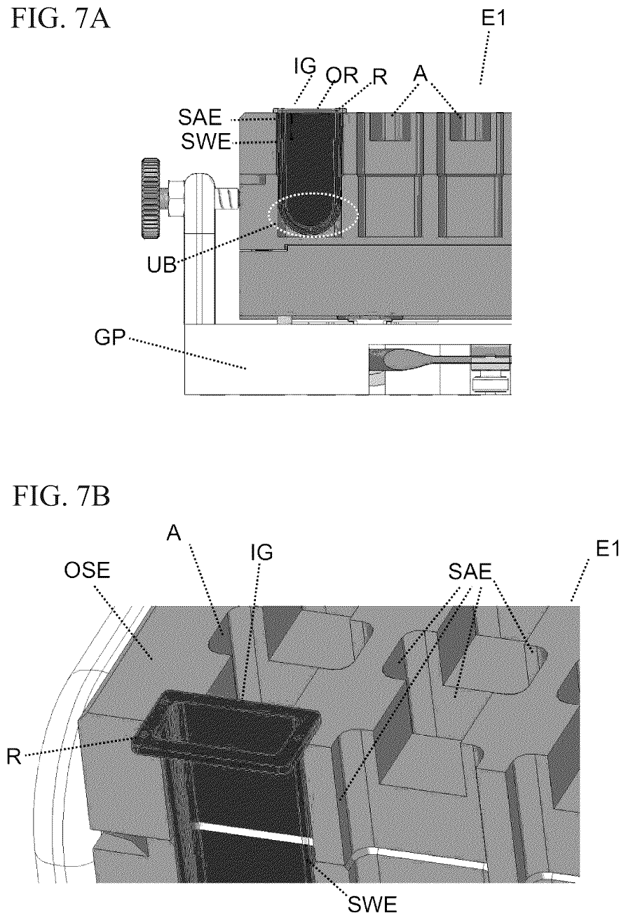

The receptacles A are intended to accommodate respective incubation vessels. A detailed view is shown in FIG. 7A in an orthogonal sectional view, visible in which are a cross section through the receptacles A and an incubation vessel IG introduced into a receptacle A. A corresponding perspective view of such a sectional view can be seen in FIG. 7B, and here too, the incubation vessel IG has been introduced into a receptacle A.

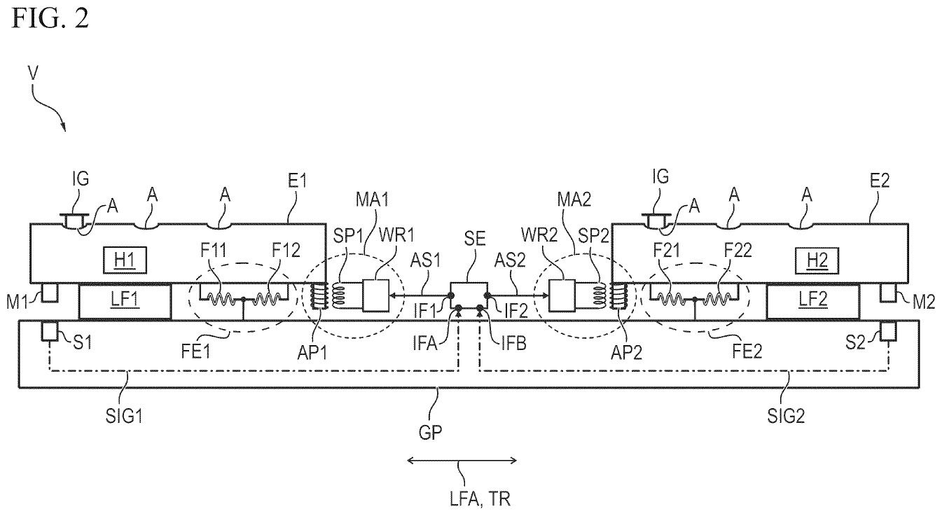

FIG. 2 shows a preferred embodiment of the device V.

The units E1, E2 are mounted on a base plate GP along a common linear guide axis LFA by means of linear guide units LF1, LF2. The units E1, E2 have respective receptacles A for respective incubation vessels IG. Preferably, the units E1, E2 have respective heating units H1, H2.

The respective units E1, E2 are mechanically coupled to the common base plate GP by means of respective spring units FE1, FE2. Here, a spring unit FE1 preferably has two spring elements F11, F12. Here, a spring unit FE2 preferably has two spring elements F21, F22.

Respective sensors S1, S2 make it possible to provide respective sensor signals SIG1, SIG2, each of which indicates one of the respective positions of the respective units E1, E2. To this end, the respective sensors S1, S2 preferably detect respective magnetic fields generated by respective magnets M1, M2.

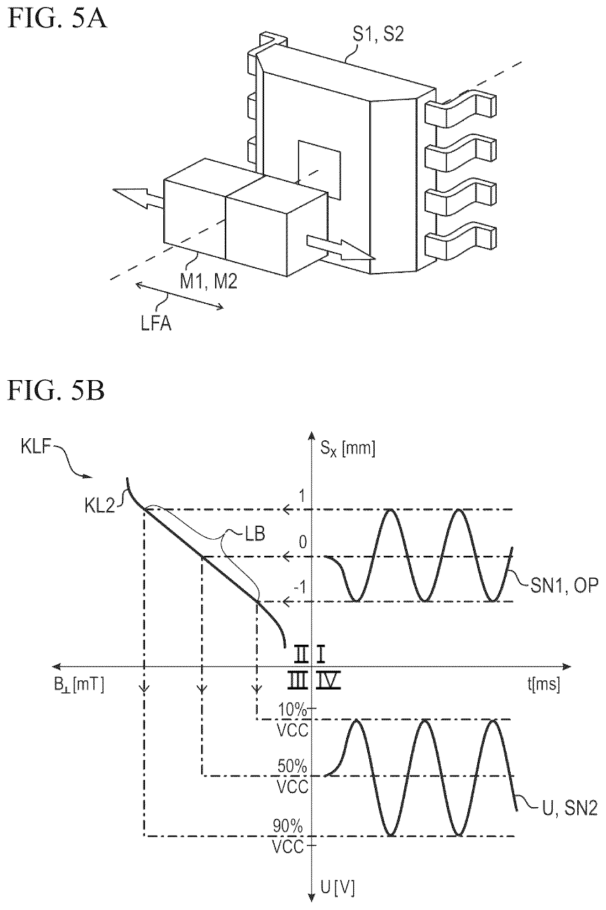

FIG. 5A shows one principle for this purpose, wherein a sensor S1, S2 is designed in particular as a Hall sensor, so that a translational movement along the linear guide axis LFA by a magnet M1, M2 can be detected. A magnet M1, M2 is in particular a diametric magnet. The magnets M1, M2 are preferably centrally aligned or positioned in the rest position with respect to the respective sensors S1, S2.

FIG. 5B shows a family of characteristics KLF, and in a top-right quadrant I, a position OP which, in the event of a mechanical oscillation, follows a corresponding sine curve SN1 is indicated in millimeters. In a top-left quadrant II, the resultant magnetic field of the transducer magnet M1, M2 is shown. When use is made of a linear region LB of the corresponding characteristic KL2 of the magnetic field, then use can be made of a linear conversion of the position OP, and so, in the bottom-left quadrant Ill, the result is then a corresponding Hall voltage which, according to the bottom-right quadrant IV, can be converted into a corresponding electrical signal or corresponding voltage U as a sine curve SN2.

Such a voltage signal U can then be provided at the control unit SE as a corresponding signal SIG1, SIG2 according to FIG. 2.

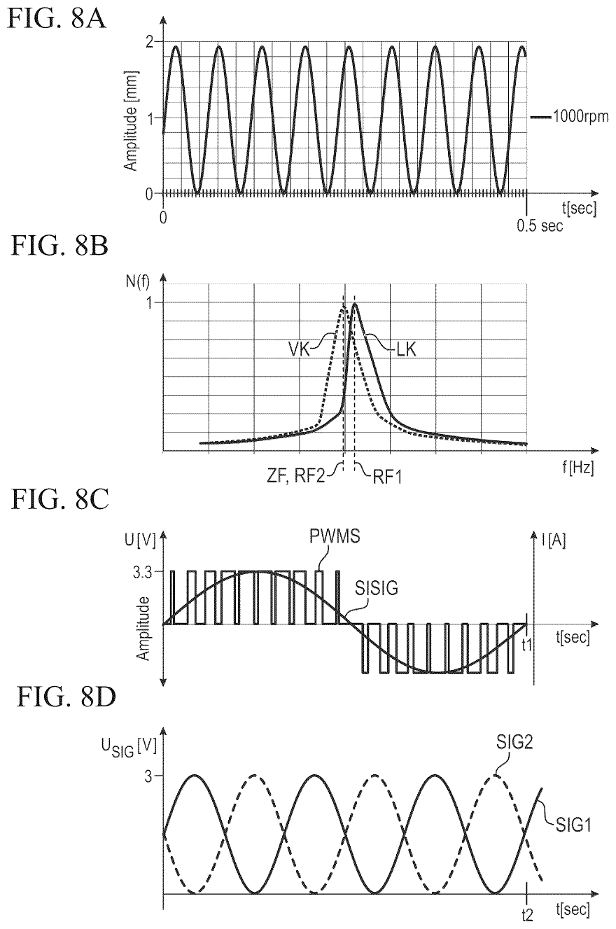

The control unit SE can control respective magnetic drive units MA1, MA2 by means of respective control signals AS1, AS2. A respective control signal AS1, AS2 is preferably respectively a pulse-width modulated signal PWMS, as shown in FIG. 8C. Here, the time in seconds is plotted on the x-axis, with time t1 preferably corresponding to a value of t1=( 1/16.67) seconds, since the oscillation frequency to be controlled is preferably 16.67 Hz. The voltage U of the pulse-width modulated signal PWMS is plotted on the y-axis. Such a signal PWMS is present at an output or interface of the control unit, which preferably comprises a microcontroller. The maximum signal level of such a pulse-width modulated signal PWMS is preferably 3.3 volts.

If such a pulse-width modulated signal PWMS is passed as a control signal AS1, AS2 to an inverter WR1, WR2 which is connected to a respective coil SP1, SP2, then a corresponding sinusoidal current signal SISIG arises through the coil, as shown in FIG. 8C. The current I of the current signal SISIG in amperes is also plotted on the y-axis. Such a current signal SISIG then yields a corresponding magnetic field signal in the vicinity of the coil. The pulse-width modulated signal PWMS and the current signal SISIG can have in particular a phase shift to each other.

Such a corresponding magnetic field can then simply be utilized such that a respective magnetic drive unit MA1, MA2 moves a respective magnetizable connection plate or return plate AP1, AP2 along the linear guide axis LFA. Preferably, such a magnetization of a connection plate AP1, AP2 by the magnetic drive units MA1, MA2 leads to changes of the respective positions of the respective units E1, E2. A respective unit E1, E2 thus has a respective connection plate or return plate AP1, AP2 which can be attracted or repelled by a respective magnetic drive unit MA1, MA2.

By means of a respective spring assembly FE1, FE2, a respective unit E1, E2 forms a spring-mass oscillation system, and in particular the respective units E1, E2 are mechanically coupled to a common base plate GP via the respective spring assemblies FE1, FE2. It is thus possible in principle, when the units E1, E2 are coupled to the common base plate GP via the spring units FE1, FE2, for both units E1, E2 to move along the linear guide axis LFA or translational direction TR such that their respective movements run in opposite directions or are shifted by 180 degrees to each other and these respective movements thus compensate each other. The spring units FE1, FE2 are preferably formed by compression springs F11, F12, F21, F22.

A unit E1, E2 preferably has a mass of 2.5 kg. A spring unit FE1, FE2 preferably has a spring constant of about 11 N/mm² to 12 N/mm².

Here, a unit E1, E2 preferably carries out a sinusoidal oscillation, which is shown in FIG. 8A. An oscillation amplitude or position in millimeters is plotted on the y-axis, and the time in seconds is plotted on the x-axis.

The design of a spring-mass oscillation system with magnetic drive is more energy efficient than a purely magnetically driven system. For a purely magnetically driven system without springs, control of the magnetic drives by the control unit would require more drive energy to generate an oscillation for a change of position. A spring-mass oscillation system can first be made to oscillate and then energy only has to be applied to maintain the oscillation.

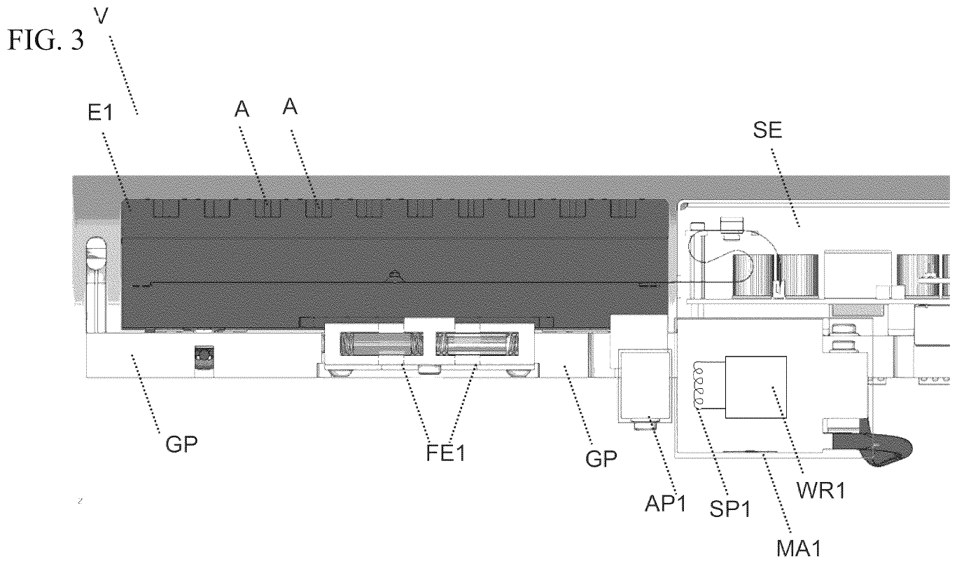

Since especially a chemiluminescence assay with bead liquid comprising magnetizable beads are present in the incubation vessels, the beads must not undergo magnetic attraction, and so it is particularly favorable to cause oscillation movement by a spring-mass oscillation system, so that there is simply no need to build up particularly high magnetic fields or forces that could have an adverse effect on positioning of the magnetizable beads in the incubation vessels. In relation to this, FIG. 3 shows again a particular sectional view of a preferred embodiment of a device V comprising a unit E1 and corresponding receptacles A, with mechanical coupling of the unit E1 to the base plate GP via the spring unit FE1. The control unit SE controls the oscillation magnet comprising a coil SP1 and an inverter WR1. The return plate AP1 can thus be influenced accordingly by means of the resultant magnetic field of the coil SP1 to influence an oscillation frequency of the unit E1. In particular, mechanical friction losses are minimized here in order to maintain the oscillation frequency or target oscillation frequency.

The units E1, E2 are preferably heatable to 37 degrees Celsius by means of the heating units H1, H2. Preferably, a unit E1, E2 has, in each case, 100 receptacles A for reaction vessels or incubation vessels IG. For thorough mixing of the reagents of the assay, the incubation plates are made to oscillate appropriately. The target frequency of such an oscillation is preferably 16 Hz to 17 Hz and the oscillation amplitude is preferably 1 mm. The magnetic beads present in the reagents or bead liquid have a higher density than a liquid in which the beads are present within the incubation vessels. Therefore, the frequency and amplitude of the oscillation prevents sedimentation of the beads, but preferably at the same time makes it possible for an incubation vessel to be picked out by a gripping unit during ongoing operation.

The proposed automated laboratory machine LA is thus preferably a so-called random access device, in which a gripping unit G during ongoing operation or during continuous oscillation of the units E1, E2 of the device V can introduce incubation vessels IG during ongoing operation into corresponding receptacles A and also pick them out again.

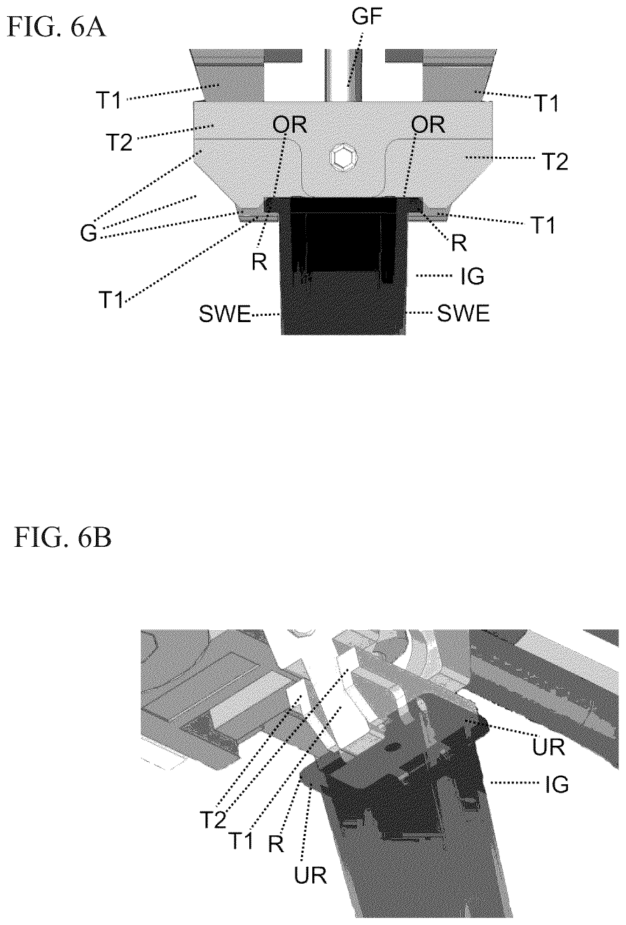

This is achieved in particular by the gripping unit being able to apply a force to a rim R of an incubation vessel IG—see FIGS. 6A and 6B—from above by a subunit T2 and from below by a subunit T1, such that only friction forces, by means of which an incubation vessel IG is held in a gripping unit G, are generated in the translational direction. In directions other than the translational direction, interlocking gripping is effected in particular. Because frictional gripping is effected in the translational direction—and simply because there is especially preferably no interlocking gripping in said direction—the incubation vessel IG can continue to carry out minimal movements in a receptacle A and in the translational direction during the gripping operation, said movements being generated in particular by movement of the units E1, E2, until especially an incubation vessel IG has been removed from the receptacle A sufficiently far from the top, until especially a sufficient distance has formed between the tapering lower region UB of the incubation vessel IG and the lateral walls SAE of the receptacle A; see also FIGS. 7A and 7B.

FIG. 8B shows a frequency response in the form of a characteristic LK for a case where a unit E1, E2 is unloaded. A frequency f in hertz is plotted on the x-axis and a normalized frequency response N(f) is plotted on the y-axis. The value N(f)=1 corresponds to a maximum value of the frequency response N(f) for the case of the unit E1, E2 being unloaded. A corresponding resonance frequency RF1 is also drawn in and is preferably f=17 Hz. Furthermore, a frequency response in the form of a characteristic VK is shown for the case of a unit E1, E2 being fully loaded with full incubation vessels. A corresponding resonance frequency RF2 or target frequency ZF is also drawn in and is preferably f=16.67 Hz.

For control of the magnetic drive units MA1, MA2 by the control unit SE, a target frequency ZF lower than the resonance frequency RF1 is provided. A corresponding characteristic is provided as the characteristic VK. The characteristic VK shows by way of example a frequency response with a resonance frequency RF2 for the case of the incubator or unit E1, E2 being fully loaded with incubation vessels of corresponding reagents.

By selecting a target frequency ZF such that the target frequency ZF is below the resonance frequency RF1 of the units E1, E2 in the unloaded state, the case is advantageously shown in the context of control or feedback control that further masses or mass elements in the form of the incubation vessels can be present in the units E1, E2 and that the necessary energy for maintaining a corresponding target frequency ZF is thus further minimized.

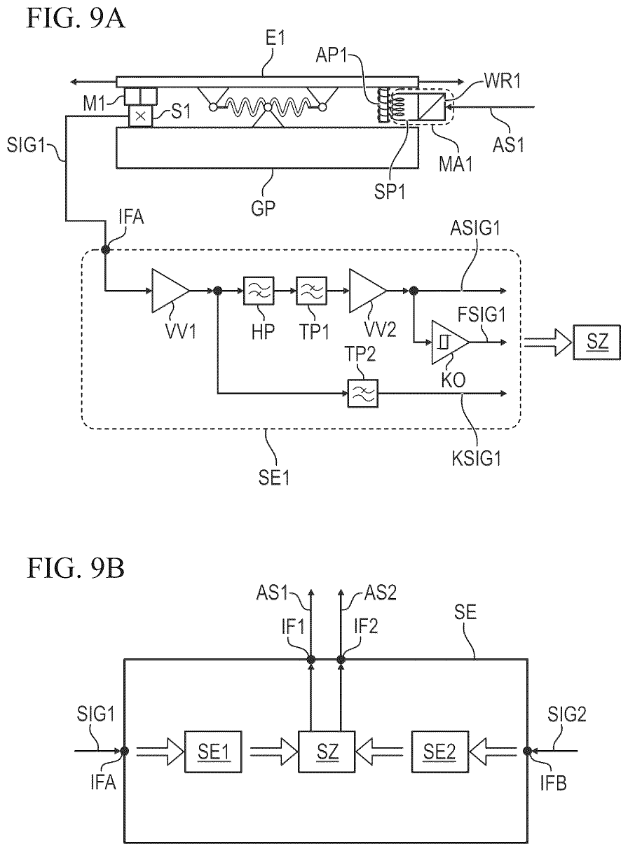

FIG. 2 shows the controller SE, which determines control signals AS1, AS2 on the basis of the sensor signals SIG1, SIG2 in order to control the magnetic drive units MA1, MA2. FIG. 9B shows in this connection a preferred embodiment of the control unit SE comprising sub-control units SE1, SE2 and SZ.

The sensor signal SIG1 is received at an interface IFA. Furthermore, the controller SE has an interface IFB for receiving the sensor signal SIG2. The sensor signal SIG1 is conditioned and processed by means of a subunit SE1. Thereafter, the determined variables or signals are provided at the central sub-control unit SZ.

The same occurs in the sub-control unit SE2 on the basis of the signal SIG2. The central sub-control unit SZ then determines the control signals AS1, AS2 and provides them at corresponding interfaces IF1 and IF2.

The control signals AS1, AS2 are pulse-width modulated signals, as shown in FIG. 8c as signal PWMS. The control signals AS1, AS2 are synchronized with respect to their duty cycle and are only modulated in terms of their pulse width.

In relation to this, FIG. 9A shows details of a sub-control unit SE1. The unit E1 is mechanically coupled to a base plate GP via the spring unit FE1 as described above. The position of the magnet M1 is monitored or detected by means of the sensor S1. The sensor S1 outputs the sensor signal SIG1 as a voltage signal. At the interface IFA, the sub-control unit SE1 receives the sensor signal SIG1. The sub-control unit SE1 derives from the sensor signal SIG1 an amplitude measurement signal ASIG1 and further preferably a frequency measurement signal FSIG1. Particularly preferably, the sub-control unit SE1 further determines on the basis of the sensor signal SIG1 a calibration signal KSIG1 to provide a signal indicating a zero position in the context of a teaching procedure before the device is finally put into operation. The sensor signal SIG1 is amplified by a preamplifier VV1. This is then preferably followed by a high pass HP, followed by a low pass TP1, thus forming a band pass. The resultant signal is then amplified by a further preamplifier VV2, thus resulting in the amplitude measurement signal ASIG1. The amplitude measurement signal can then be used to further determine preferably the frequency measurement signal FSIG1 by means of a comparator KO. The sensor signal SIG1 amplified by the preamplifier VV1 can further preferably be filtered by means of a low pass filter TP2 in order to determine the calibration signal KSIG1. The determined signals ASIG1, FSIG1, KSIG1 can then be provided at the central sub-control unit SZ. Preferably, at least the determined signals ASIG1, amplitude signal, and FSIG1, frequency signal, are provided at the central sub-control unit SZ.

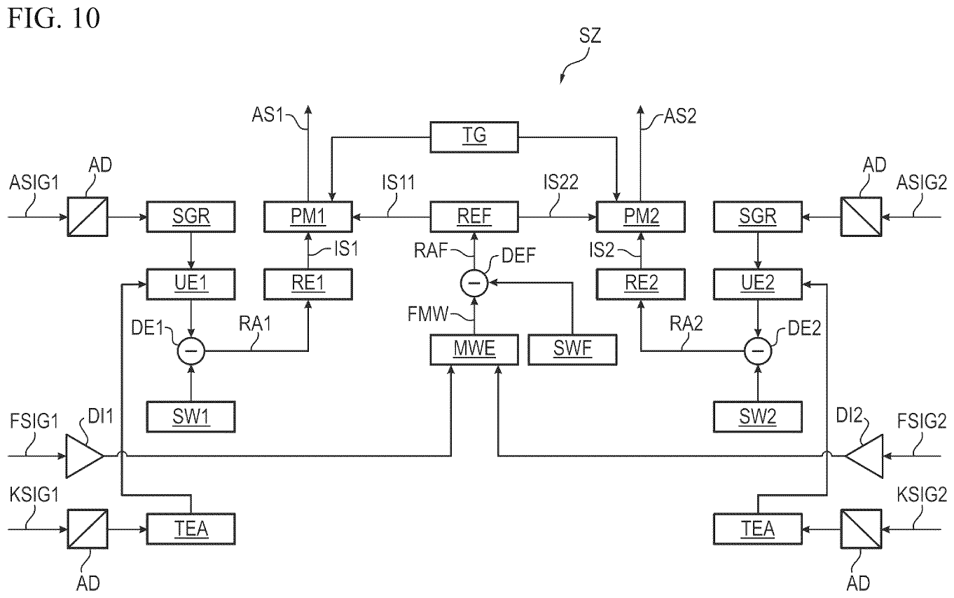

The sub-control unit SE2 from FIG. 9B determines on the basis of the second sensor signal SIG2 corresponding signals in the form of an amplitude measurement signal, a frequency measurement signal and preferably a calibration signal and also provides them at the central sub-control unit SZ. Further details can be found in FIG. 10, which illustrates one embodiment of the sub-control unit SZ.

The control signals AS1, AS2 are determined so that the resultant positions or oscillation amplitudes of the two moving units behave as shown by corresponding signals SIG1, SIG2 in FIG. 8D. Time is plotted on the x-axis, with preferably time t2 corresponding to a value of t1=( 3/16.67) seconds, since the oscillation frequency to be controlled is preferably 16.67 Hz. The sensor voltage USIG in volts is plotted on the y-axis. The resultant respective oscillation frequencies of the respective units then have a phase shift of 180 degrees to each other. To this end, the control unit determines using the sub-control unit SE1, i.e., on the basis of the sensor signal SIG1, an amplitude measurement signal or amplitude ASIG1 and an oscillation frequency or frequency measurement signal FSIG1. Furthermore, the sub-control unit SE2 determines on the basis of the sensor signal SIG2 an amplitude measurement signal or amplitude ASIG2 and an oscillation frequency or frequency measurement signal FSIG2 and preferably a calibration signal KSIG2. The control unit then determines, especially by means of the sub-control unit SZ, the control signals AS1, AS2 in such a way that both units are adjusted to a common oscillation amplitude and a common, identical target oscillation frequency. In addition, the oscillation frequencies have in particular a phase shift of 180 degrees to each other. The control carried out by the control unit SE is a feedback control, in particular a PI control, based on a common, identical target oscillation frequency.

Following an analog-to-digital conversion by an AD converter, the amplitude measurement signal ASIG1 is then rectified by means of a peak value rectifier SGR. A conversion unit UE1 then converts the digital signal into millimeters. A difference-forming unit DE1 receives a setpoint of preferably 1 mm from a setpoint unit SW1 and then determines as the difference the control deviation RA1, which is supplied to a feedback control unit RE1. The feedback control unit RE1 carries out a PI control, the values used thereby being preferably a KP value of 0.03 and a KI value of 0.6. A corresponding determination of a control deviation RA2 on the basis of the sensor signal ASIG2 is done by means of an AD converter AD, a peak value rectifier SGR and a conversion unit UE2, and here too, the setpoint, which is specified by the setpoint unit SW2, is 1 mm, and so the difference-forming unit DE2 determines the control deviation RA2. A PI control in the feedback control unit RE2 is then carried out using the same KP and KI values of the unit RE1.

The frequency measurement signal FSIG1 is provided at an averaging unit MWE via a digital input D11. The same occurs or is done by supplying the frequency measurement signal FSIG2 via a digital input D12 and providing it at the averaging unit MWE. A setpoint unit SWF provides a setpoint of preferably 16.67 Hz, and so a difference-forming unit DEF then determines a difference between the average frequency value FMW and the setpoint as a control deviation RAF. A feedback control unit REF determines, on the basis of the input signal or the control deviation RAF by means of a PI control and parameter values of KP=0.009 and KI=0.09, a PI control in such a way that respective internal control signals IS11 and IS22 are provided at the respective modulators PM1 and PM2.

A synchronous clock generator TG controls corresponding pulse-width modulators PM1 and PM2. The pulse-width modulator PM1 is controlled with respect to its pulse width by a signal IS1 of the amplitude control unit RE1 and a signal IS11 of the frequency control unit REF and then generates the control signal AS1. The pulse-width modulator PM2 is controlled with respect to its pulse width by a signal IS2 of the amplitude control unit RE2 and a signal IS22 of the frequency control unit REF and then generates the control signal AS2.

As already explained above, a detailed view of FIG. 7A shows an orthogonal sectional view, visible in which are a cross section through the receptacles A and an incubation vessel IG introduced into a receptacle A. A corresponding perspective view of such a sectional view can be seen in FIG. 7B, and here too, the incubation vessel IG has been introduced into a receptacle A.

The incubation vessel IG preferably converges in a tapered manner in a lower region UB and has in its upper region a circumferential rim R which projects above the lateral walls SWE of the vessel.

When the incubation vessel IG is introduced into a receptacle A, the rim R thereof comes to rest on an upper face OSE of the unit E1. As a result, the incubation vessel IG is at least partially held in the receptacle A. Furthermore, the incubation vessel IG is preferably held in that there is at least partially an interlock between the receptacle A and the incubation vessel IG.

FIG. 6A shows an incubation vessel IG with its rim R and the lateral walls SWE, the incubation vessel IG being held by a gripping unit G. Such a representation is also shown in FIG. 6B in an oblique view.

A first part T1 of a clamping mechanism of the gripping unit G grasps the incubation vessel IG on two sides below the rim R or on the lower face UR of the rim. Furthermore, a second subunit T2 of a clamping mechanism of the gripping unit G grips the incubation vessel in that said second subunit T2 is pressed from above by a spring unit GF onto the upper face OR of the rim of the incubation vessel IG. The subunits T1 and T2 of the gripping unit thus interact to generate a friction force for holding the incubation vessel. As a result, the gripping unit G can grip the incubation vessel IG.

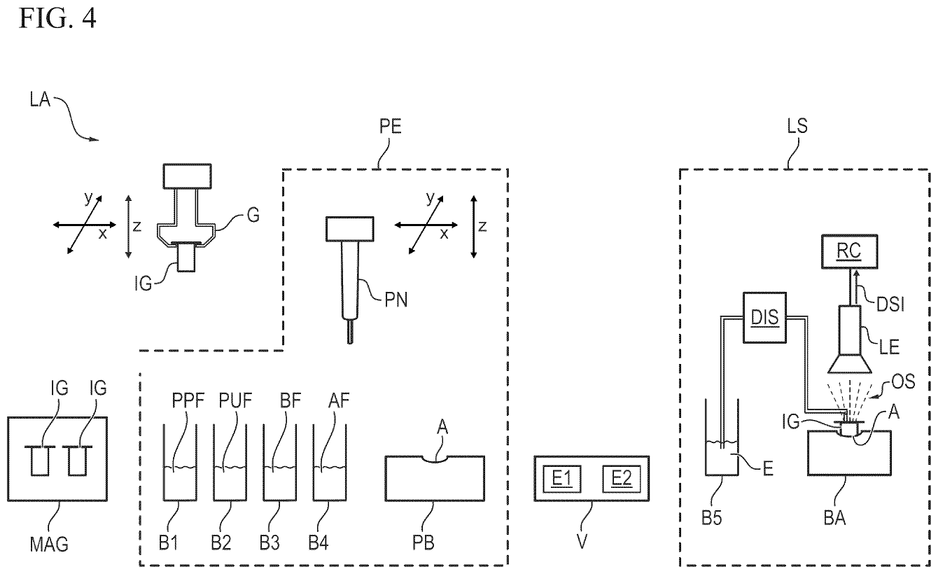

FIG. 4 shows a schematic diagram of a preferred embodiment of an automated laboratory machine LA according to the invention. The automated laboratory machine LA comprises a gripping unit G for gripping incubation vessels IG. The automated laboratory machine LA further comprises a magazine MAG containing multiple incubation vessels IG. By means of the gripping unit G, incubation vessels IG can be transferred from the magazine MAG to a pipetting unit PE. Provided in a preferably provided pipetting base PB of the pipetting unit PE is then a receptacle A for accommodating an incubation vessel IG. The pipetting unit PE can thus in particular accommodate one incubation vessel IG in one receptacle A.

The automated laboratory machine LA further comprises a container B1 containing a patient sample liquid PPF. The automated laboratory machine LA further comprises a container B2 containing a buffer liquid PUF. The automated laboratory machine LA further comprises a container B3 containing a bead liquid BF. The automated laboratory machine further comprises a container B4 containing a label liquid AF comprising antibodies labeled with a chemiluminescence label or antigens labeled with a chemiluminescence label.

The automated laboratory machine LA further comprises a device V according to the invention.

The gripping unit G is thus further designed to bring incubation vessels IG from the pipetting unit PE to respective receptacles of the respective units E1, E2 of the device V and also to remove them again, in particular in order also to return incubation vessels from the device V to the pipetting unit PE. For this purpose, the gripping unit G is preferably displaceable or movable in the X direction, Y direction and Z direction. The gripping unit G can grip incubation vessels IG and also release them again or deposit them in an automated manner.

The pipetting unit PE comprises one or more pipetting needles PN which are preferably movable or displaceable in the X direction, Y direction and Z direction. By means of such pipetting needles PN, various liquids of the liquids PPF, PUF, WF, AF can be pipetted from the containers B1, B2, B3, B4 into an incubation vessel IG or be aspirated therefrom.

To carry out a chemiluminescence assay, the automated laboratory machine LA further preferably comprises a reading station LS. Thus, the automated laboratory machine LA further preferably comprises a container B5 containing a liquid or trigger liquid E, which in turn comprises an enzyme for implementation in the chemiluminescence reaction.

Preferably, a dispensing unit DIS introduces the enzyme E into an incubation vessel IG which is preferably present in a receptacle A of a base BA. Resultant optical radiation or a resultant optical signal OS can preferably be detected by means of a reading unit LE. Preferably, after the optical signal OS has been detected, a corresponding digital signal DSI can then be evaluated by a computation unit RC and be preferably transmitted via a data interface of the computation unit RC via a data network. The computation unit RC is a preferably integral part of the automated laboratory machine LA. The reading station LS thus comprises the reading unit LE and the computation unit RC.

The automated laboratory machine is in particular an automated laboratory machine for processing a patient sample using a chemiluminescence assay for detection of a target antigen or a target antibody in the patient sample.

The patient sample is in particular a liquid patient sample. The patient sample liquid can have a homogeneous liquid phase. Preferably, the patient sample liquid is human samples that have been collected for diagnostic testing and optionally prepared, preferred examples being blood, preferably blood serum, urine, cerebrospinal fluid, saliva or sweat.

For many automated systems, so-called chemiluminescence methods or chemiluminescence assays are used for detection of a specific antigen or target antigen or a specific antibody or target antibody in a patient sample in order to gain knowledge of a possible health state of a patient. In particular, use is made here of beads as carriers for reagents. For example, the beads can be carriers for immobilized antigens, and antibodies or target antibodies to be detected in human samples bind to said antigens. The beads are usually supplied in aqueous solutions, the so-called bead liquid, and stored in said solutions until use. The beads are in particular magnetizable particles, in particular metallic magnetizable particles. If such beads are incubated with a liquid patient sample and if the target antibodies are present, then what is formed is an antigen-antibody complex that is immobilized on the bead. After a wash step with a rinse liquid or buffer liquid, said complex can be incubated with suitable reagents, referred to in particular as conjugate. These are in particular label liquids comprising secondary antibodies labeled with a chemiluminescence label, particularly preferably in the form of acridinium. Said secondary antibody is preferably an anti-human antibody, preferably anti-human IgG antibody. This principle forms the basis of the so-called automated chemiluminescence machines that are commercially available and that can preferably also be a random access analyzer. To generate an optical signal, a solution or trigger solution containing an enzyme is then added at the end of execution of the chemiluminescence method in order to cause a chemiluminescence reaction between the enzyme and the chemiluminescence label, such that an optical signal is produced. The resultant optical signal is proportional to the antibody concentration in the patient sample and, after detection, can preferably be automatically converted into a concentration by the automated laboratory machine. The label liquid is in particular also referred to as conjugate liquid.

To detect an antigen instead of an antibody, antibody-coated magnetic particles, known as beads, are incubated with the patient sample and an antigen-specific biotinylated antibody. During incubation, the antigen is bound by both the magnetic particle-coupled antibody and the biotinylated antibody. In a further step, conjugate is added and it binds to the biotinylated antibody. The reaction is then mixed with the trigger solution, which induces a chemiluminescence reaction.

Immunodiagnostic tests or assays working according to this principle are described for numerous indications in the prior art, for example in EP 2 199 303, DE 10 2009033281, WO 2010/009457 or EP12183919.5. Principles of a chemiluminescence method are also described in EP 3 160 646 B1.

Suitable reagents are described in the prior art, for example in Ireland, D., and Samuel, D. (1989), “Enhanced Chemiluminscence ELISA for the detection for antibody to hepatitis B virus surface antigen”, J. Biolum. Chemilumin., 159-163 and in “Acridinium Esters as Highly Specific Activity Labels in Immunoassays”, Clin. Chemistry 19:1474-1478 (1984) and in U.S. Pat. No. 4,842,997 A.

A particular problem is inhomogeneous bead liquids, for example suspensions of beads in aqueous solution, the density of which is higher than that of water, meaning that the beads can sink to the bottom. In the course of incubation, sedimentation of the beads may occur in an incubation vessel. Alternatively, such beads in a liquid phase may readily accumulate on surfaces. Therefore, the incubation vessels are moved or made to oscillate in a regular manner during incubation.

The bead liquid may alternatively have an inhomogeneous phase and comprise either two immiscible or only slightly miscible liquids or a solid material in a liquid. In a preferred embodiment, the liquid is beads in an aqueous solution. Such beads can be provided with biological reagents immobilized thereon, for example polypeptides acting as antigen. Commercially, various beads, mainly carbohydrate-based (e.g., agarose) or plastic-based, are available for numerous applications. They contain active or activatable chemical groups, such as a carboxyl group, that can be utilized for immobilization of reagents, for example antibodies or antigens. Preferably, the beads are beads having an average diameter of 0.2 mm to 5 mm, 0.5 mm to 1 mm, 0.75 mm to 100 mm or 1 mm to 10 mm. The beads can be coated with an antigen that binds to a diagnostically relevant antibody, or with affinity ligands, for example biotin or glutathione. Preferably, the liquid comprises the beads in the form of an aqueous suspension having a bead content of 10% to 90%, more preferably 20% to 80%, more preferably 30% to 70% and even more preferably 40% to 60% (w/w).

In a particularly preferred embodiment, the beads are paramagnetic beads that can be readily concentrated on a surface with the aid of a magnet. For this purpose, commercially available paramagnetic beads usually contain a paramagnetic mineral, for example iron oxide.

The buffer liquid can also be referred to as rinse liquid. Such buffer liquids or rinse liquids are known to a person skilled in the art, in particular from EP 3 160 646 B1.

The features disclosed in the description and the drawings may be relevant either individually or in any combination for realization of exemplary embodiments in their various configurations and—unless indicated otherwise in the description—may be combined with each other as desired. Although some aspects have been described in connection with a device, especially in the form of an automated laboratory machine, it is understood that said aspects are also a description of the corresponding method, and so a block or a component of a device can also be understood as a corresponding method step or as a feature of a method step. By analogy, aspects which have been described in connection with a method step or as a method step are also a description of a corresponding block or detail or feature of a corresponding device or automated laboratory machine.

Depending on specific implementation requirements, exemplary embodiments of the invention can be implemented in hardware or in software, in particular a control unit and/or a reading unit and/or a reading station. A programmable hardware component can in particular be formed by a microcontroller, a processor, a central processing unit (CPU), a graphics processing unit (GPU), a computer, a computer system, an application-specific integrated circuit (ASIC), an integrated circuit (IC), a system on a chip (SOC), a programmable logic element or a field-programmable gate array with a microprocessor (FPGA) or just an FPGA without a microcontroller.

1. A device for incubating multiple patient samples using multiple incubation vessels, comprising:
a first unit (E1) having respective receptacles (A) for respective incubation vessels (IG),
and a second unit (E2) having respective receptacles (A) for respective incubation vessels (IG),
where both of the respective units (E1, E2) are each movably mounted in a respective position along a common linear guide axis (LFA),
further comprising
respective sensors (S1, S2) for providing respective sensor signals (SIG1, SIG2), each of which indicates one of the respective positions,
furthermore respective magnetic drive units (MA1, MA2) for respective changes of the respective positions,
and furthermore a control unit (SE) designed to control the respective magnetic drive units (MA1, MA2) on the basis of the respective sensor signals (SIG1, SIG2) to cause the respective changes of the respective positions.

2. The device as claimed in claim 1,
wherein the two units (E1, E2) are each mounted and guided along the common linear guide axis (LFA) in such a way that they have only one common translational degree of freedom.

3. The device as claimed in claim 1,
wherein a respective unit (E1, E2) is mechanically coupled to a common base plate (GP) via a respective spring unit (FE1, FE2).

4. The device as claimed in claim 3,
wherein a respective unit (E1, E2) is mechanically coupled to the common base plate (GP) via a respective spring unit (FE1, FE2) such that the respective unit (E1, E2) together with the respective spring unit (FE1, FE2) forms a respective spring-mass oscillation system.

5. The device as claimed in claim 1,
wherein the control unit (SE) is designed to control the magnetic drive units (MA1, MA2) such that the units (E1, E2) are each maintained in oscillation with a respectively substantially identical oscillation amplitude in a direction of the linear guide axis (LFA).

6. The device as claimed in claim 1,
wherein the control unit (SE) is designed to control the magnetic drive units (MA1, MA2) by means of feedback control on the basis of the sensor signals (SIG1, SIG2) such that resultant respective oscillation frequencies of the respective units are substantially identical and that the respective oscillation frequencies have a phase shift of substantially 180 degrees to each other.

7. The device as claimed in claim 1,
wherein the control unit (SE) is designed to determine a respective current amplitude and a respective oscillation frequency for a respective unit (E1, E2) on the basis of a respective sensor signal (SIG1, SIG2),
and wherein the control unit (SE) is designed to adjust both units (E1, E2) to a common oscillation amplitude and a common, identical target oscillation frequency.

8. The device as claimed in claim 3,
wherein both units (E1, E2) with the respective spring units (FE1, FE2) each comprise a spring-mass oscillation system with a respectively identical resonance frequency (RF1) in an unloaded state
and wherein a target oscillation frequency is below said resonance frequency (RF1).

9. The device as claimed in claim 1,
wherein the control unit (SE) is designed to perform a PI control based on a common, identical target oscillation frequency.

10. The device as claimed in claim 1,
wherein both units (E1, E2) have a respective heating unit (H1, H2).

11. An automated laboratory machine (LA) comprising a device (V) as claimed in claim 1, further comprising:
a magazine (MAG) containing a plurality of incubation vessels (IG),
a container (B1) containing a patient sample liquid (PPF), a container (B2) containing a buffer liquid (PUF), a container (B3) containing a bead liquid (BF) comprising beads coated with antigens or with antibodies, and a container (B4) containing a label liquid (AF) comprising antibodies or antigens labeled with a chemiluminescence label,
a pipetting unit (PE) designed to dispense at least a portion of the patient sample liquid, at least a portion of the buffer liquid, at least a portion of the bead liquid and at least a portion of the label liquid into the incubation vessels and to aspirate them from the incubation vessels,
and furthermore a gripping unit (G) designed to transfer incubation vessels
from the magazine (MAG) to the pipetting unit (PE)
and to transfer them from the pipetting unit (PE) to the respective receptacles (A), to remove them from the respective receptacles (A) and to transfer them back to the pipetting unit (PE).

12. The automated laboratory machine as claimed in claim 11,
further comprising a container (B5) containing a liquid comprising an enzyme (E) for implementation of a chemiluminescence reaction,
and further comprising a reading unit (LE) for detecting an optical signal (OS) of a chemiluminescence reaction.

13. The automated laboratory machine as claimed in claim 11,
wherein the magnetic drives (MA1, MA2) are arranged such that beads present in the bead liquid (BF) are not substantially influenced magnetically by magnetic fields of the magnetic drives (MA1, MA2).

14. The automated laboratory machine as claimed in claim 11, further comprising:
a container (B5) containing a liquid comprising an enzyme (E) for implementation of a chemiluminescence reaction,
a dispensing unit for introducing the liquid comprising the enzyme into an incubation vessel, and
a reading unit (LE) for detecting an optical signal (OS) of a chemiluminescence reaction.
